(19) United States
(12) Patent Application Publication
(10) Pub. No.: US 2021/0388372 A1
(43) Pub. Date: Dec. 16, 2021

(54) NOVEL GENE RELATED TO PLANT DROUGHT STRESS TOLERANCE AND USES THEREOF

(71) Applicant: UIF (UNIVERSITY INDUSTRY FOUNDATION),YONSEI UNIVERSITY, Seodaemun-gu (KR)

(72) Inventors: Woo Taek KIM, Seoul (KR); Seong Gwan YU, Gyeonggi-do (KR); Na Hyun CHO, Jeollabuk-do (KR); Jong Hum KIM, Seoul (KR); Tae Rin OH, Gyeonnggi-do (KR)

(21) Appl. No.: 17/345,567

(22) Filed: Jun. 11, 2021

(30) Foreign Application Priority Data
Jun. 11, 2020 (KR) 10-2020-0071134

(51) Int. Cl. C12N 15/82; C12N 9/10

(52) U.S. Cl. CPC C12N 15/8273; C12N 9/104

(57) ABSTRACT

Disclosed herein are a novel gene for improving drought stress tolerance in plants and a use thereof. Specifically, disclosed is a composition for improving the drought stress tolerance of plants. The composition includes drought responsive ring 1 (DRR1) protein or a gene sequence that encodes the protein. Further disclosed are a plant cell and a plant either of which is transformed with the composition. The composition improves the tolerance of a plant to drought stress. The transformed plant cell or plant has excellent tolerance to drought stress and thus can be usefully used as a novel functional crop that can reduce the loss of crop yield caused by dry environmental stress in the cultivation stage of the plant.

### CROSS REFERENCE TO RELATED APPLICATION

The present application claims priority to Korean Patent Application No. 2020-0071134, filed Jun. 11, 2020, the entire contents of which is incorporated herein for all purposes by this reference.

### BACKGROUND OF THE INVENTION

### 1. Field of the Invention

The present invention relates to a novel gene involved in drought stress tolerance in plants and a use thereof. Morea specifically, the present invention relates to a composition for improving drought stress tolerance in plants, the composition including drought responsive ring 1 (DRR1) and a gene sequence encoding the protein, and a plant cell and a plant transformed with the composition.

### 2. Description of the Related Art

As living areas have increased due to increasing population, available arable land has decreased, and climate change is proceeding in real time as a result of industrialization to support the population. As a result, crop production has decreased and it is likely to lead to a problem of food shortage in the near future.

In crop production, due to immobility, plants face various environmental factors (stresses such as drought, high salt, heavy metals, cold damage, thermal shock, and ozone) throughout their lifetime. Since such environmental stresses become limiting factors for the growth and development of crops, it is important to study the functions of stress response genes to increase crop productivity. Therefore, in order to increase the production rate of plants, studies on improving the tolerance of plants to stress are being actively conducted.

Meanwhile, ubiquitin, which is a protein consisting of 76 amino acids expressed in all eukaryotes, has a characteristic in that it covalently binds to various substrate proteins by a E1-E2-E3 (ubiquitin activation, ubiquitin binding, ubiquitin linking enzyme) chain enzyme reaction. Studies have well identified that the matrix protein to which ubiquitin is attached is so diverse that it affects almost all physiological activities in cells and that many diseases are associated with this mechanism. The main function of ubiquitin identified thus far was to promote protein degradation by binding to other proteins, but other functions of ubiquitin have recently been revealed one after another.

In particular, E3 ubiquitin (Ub) ligase is a protein that induces degradation of a corresponding protein through the 26S proteasome system by linking ubiquitin, which is a small labeling protein well preserved in all living organisms, to a specific substrate, and it has been reported that various E3 Ub ligases are involved in the drought stress tolerance mechanism through the regulation of the amounts and functions of various intracellular proteins including transcription factors that regulate gene expression under drought stress in plants (Ryu et al., 2010; Cho et al., 2011).

According to recent studies, insoluble proteins tend to accumulate in plants under drought stress, and thus there is an increasing need for studies relating to the removal of these insoluble proteins. However, not much progress has been made yet with regard to identification of the relationship between the accumulation of insoluble proteins and drought stress, and the knowledge of the biological functions of genes involved in tolerance or sensitivity to drought stress is still insufficient.

### DOCUMENTS OF RELATED ART

### Non-Patent Documents

(Non-Patent Document 1) Ryu et al. (2010), Plant physiology 154: 1983-1997

(Non-Patent Document 2) Cho et al. (2011), Plant physiology 157: 2240-2257

### SUMMARY OF THE INVENTION

Accordingly, in an effort to solve the above problems occurring in the prior art, the present inventors have conducted extensive research to develop a method for improving crop productivity in a drought stress environment. As a result, they have confirmed that the tolerance of plants to drought stress can be changed by selecting drought responsive ring 1 (DRR1), which is a novel RING-type E3 ligase involved in drought stress tolerance, by controlling the amount of insoluble proteins accumulated in Arabidopsis under dry conditions; and controlling the function of the DRR1 gene, thereby completing the present invention.

Accordingly, an objective of the present invention is to provide a composition for improving drought stress tolerance in plants, in which the composition includes drought responsive ring 1 (DRR1) protein or a gene sequence encoding the protein.

Additionally, another objective of the present invention is to provide a plant cell or plant having improved tolerance to drought stress, in which the plant cell or plant is transformed with the composition.

Additionally, still another objective of the present invention is to provide a method for improving tolerance of a plant to drought stress, in which the method includes introducing the composition into a plant cell.

However, the technical problems to be achieved by the present invention are not limited to those described above, and still other problems not described will be clearly understood by those skilled in the art from the following description.

In order to achieve the objectives described above, the present invention provides a composition for improving drought stress tolerance in plants, in which the composition contains drought responsive ring 1 (DRR1) protein or a gene sequence that encodes the protein

In an embodiment of the present invention, the composition may include an amino acid sequence of SEQ ID NO: 1.

In another embodiment of the present invention, the gene sequence encoding the drought responsive ring 1 (DRR1) protein may include a nucleotide sequence of SEQ ID NO: 2.

In still another embodiment of the present invention, the composition can inhibit the accumulation of insoluble proteins in plants.

Additionally, the present invention provides a plant cell or plant having improved tolerance to drought stress, in which the plant cell or plant is transformed with the composition.

Additionally, the present invention provides a method for improving tolerance of a plant to drought stress, in which the method includes introducing the composition into a plant cell.

The composition of the present invention for improving drought stress tolerance in plants improves the tolerance of a plant to drought stress, and the transformed plant cell or plant of the present invention has excellent tolerance to drought stress and thus can be usefully used as a novel functional crop that can reduce the loss of crop yield caused by dry environmental stress in the cultivation stage of the plant.

### BRIEF DESCRIPTION OF THE DRAWINGS

The above and other objectives, features, and other advantages of the present invention will be more clearly understood from the following detailed description taken in conjunction with the accompanying drawings, in which:

FIG. 1 shows a CDS schematic diagram of DRR1 gene;

FIG. 2 shows the results of RT-PCR examination of the mRNA expression level of the DRR1 gene;

FIG. 3 shows the results of co-localization with BiP1-mRFP-HDEL protein, which is an endoplasmic reticulum marker in plants;

FIG. 4 shows the results of a fractionation assay of DRR1 protein using a tobacco expression system;

FIG. 5 shows the results of confirming the presence/absence of the loss of DRR1 function in a plant (Cas9:DRR1) with a loss of DRR1 function based on the CRISPR-Cas9 technique;

FIG. 6 shows the results of RT-PCR of a plant (DRR1: RNAi) with a reduced function based on the RNAi technique of DRR1.

FIG. 7 shows the examination results of drought stress tolerance of a plant (Cas9:DRR1) with a loss of DRR1 function based on the CRISPR-Cas9 technique and plants (DRR1: RNAi #1, #2) with a reduced function based on the RNAi technique of DRR1;

FIG. 8 shows the changes in the amount of insoluble proteins accumulated under a drought stress condition of a plant (Cas9:DRR1) with a loss of DRR1 function based on the CRISPR-Cas9 technique and plants (DRR1: RNAi #1, #2) with a reduced function based on the RNAi technique of DRR1; and

FIG. 9 shows the examination results of insoluble abnormal protein-induced stress tolerance of a plant (Cas9:DRR1) with a loss of DRR1 function based on the CRISPR-Cas9 technique and plants (DRR1: RNAi #1, #2) with a reduced function based on the RNAi technique of DRR1.

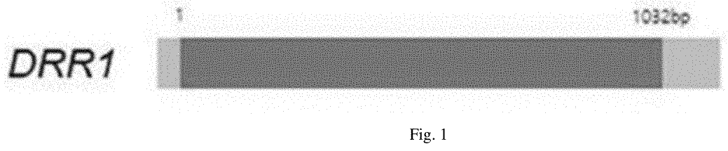

### DESCRIPTION OF THE PREFERRED EMBODIMENTS

The present inventors have confirmed that the tolerance of plants to drought stress can be changed by selecting drought responsive ring 1 (DRR1), which is a novel RING-type E3 ligase involved in drought stress tolerance, by controlling the amount of insoluble proteins accumulated in Arabidopsis under dry conditions; and controlling the function of the DRR1 gene, thereby completing the present invention.

As such, the present invention provides a composition for improving drought stress tolerance in plants, in which the composition includes drought responsive ring 1 (DRR1) protein or a gene sequence encoding the protein.

As used herein, the term “drought stress” refers to the stress of a plant caused by humidity that is lower than the normal growing environment or by the lack of moisture, and the improvement of tolerance of a plant to drought stress in the present invention means that the plant has a significantly higher survival rate by minimizing moisture loss of the plant under dry conditions.

According to an embodiment of the present invention, the drought responsive ring 1 (DRR1) protein may include an amino acid sequence of SEQ ID NO: 1, and the gene encoding the DRR1 protein may include a nucleotide sequence of SEQ ID NO: 2.

It is apparent to those skilled in the art that the nucleotide sequences used in the present invention are not limited to the ones shown in the accompanying sequence listing.

A mutation in a nucleotide may not necessarily result in a change in a given protein. Such a nucleic acid includes a nucleic acid molecule including a functionally equivalent codon, or a codon encoding the same amino acid (e.g., by the degeneracy of codons, there are six codons for arginine or serine), or a codon encoding a biologically equivalent amino acid.

Considering the mutations having the biologically equivalent activity described above, the DRR1 protein (SEQ ID NO: 1) with drought stress tolerance is interpreted as also including a sequence exhibiting substantial identity to the sequence described in SEQ ID NO. The exhibition of the substantially identity means that the drought responsive ring 1 (DDR1) consists of an amino acid sequence that has a sequence homology of at least 70%, preferably at least 80%, more preferably at least 90%, and most preferably at least 95, 96%, 97%, 98%, or 99% to SEQ ID NO, in the case where the sequence of the present invention and any other sequences are aligned so that they can maximally correspond to each other, and the aligned sequences are analyzing using an algorithm commonly used in the art.

As used herein, the term “plant” refers not only to a mature plant but also to all of a plant cell, a plant tissue, a plant seed, etc. that can be developed into a mature plant.

In the present invention, the plant is not particularly limited. As the plant according to the present invention, most dicotyledonous plants or monocotyledonous plants may be both used, and preferably may be applicable to a plant selected from the group consisting of food crops including rice, wheat, barley, corn, soybean, potato, wheat, red bean, oat, and sorghum; vegetable crops including Arabidopsis, Chinese cabbage, radish, red pepper, strawberry, tomato, watermelon, cucumber, cabbage, melon, pumpkin, green onion, onion, and carrot; special crops including ginseng, tobacco, cotton, sesame, sugar cane, sugar beet, perilla, peanut, and rapeseed; fruit trees including apple trees, pear trees, dates, peaches, poplars, grapes, tangerines, persimmons, plums, apricots, and bananas; flowers including roses, gladiolus, Gerbera, carnations, chrysanthemums, lilies, and tulips; and forage crops including ryegrass, red clover, orchard grass, alpha-alpha, tall fescue, and perennial ryegrass. The composition according to the present invention can control drought stress tolerance by inhibiting the accumulation of insoluble proteins in plants.

The present inventors have confirmed through specific examples that the DRR1 gene has tolerance to drought stress and controls drought stress tolerance by inhibiting the accumulation of insoluble proteins in plants.

More specifically, in an embodiment of the present invention, in order to prepare a plant in which the function of the DRR1 gene is lost or reduced, a plant (Cas9:DRR1) in which the DRR1 gene sequence is modified was prepared based on the CRISPR-Cas9 technique thereby confirming that the plant lost the function of the DRR1 gene; and a plant (DRR1:RNAi) in which the DRR1 gene sequence is modified was prepared based on the RNAi technique thereby confirming that the plant has a reduced function of the DRR1 gene (see Example 4).

In another embodiment of the present invention, as a result of examining the drought stress tolerance of the plant (Cas:DRR1) in which the function of the DRR1 gene is lost or the plants (DRR1-RNAi #1, #2) in which the function of the DRR1 gene is reduced, it was confirmed that the survival rates of the plant (Cas:DRR1) in which the function of the DRR1 gene is lost and the plants (DRR1-RNAi #1, #2) in which the function of the DRR1 gene is reduced were significantly lower (see Example 5).

In still another embodiment of the present invention, as a result of analyzing the changes in the amount of accumulation of an insoluble protein (ubiquitinated protein) after extracting proteins from the plant (Cas:DRR1) in which the function of the DRR1 gene is lost and the plants (DRR1-RNAi #1, #2) in which the function of the DRR1 gene is reduced, followed by centrifugation, it was confirmed that the amount of accumulation of the ubiquitinated insoluble protein was increased in the plant (Cas:DRR1) in which the function of the DRR1 gene is lost and the plants (DRR1-RNAi #1, #2) in which the function of the DRR1 gene is reduced compared to the wild-type (WT), which is the control group (see Example 6); and as a result of analyzing the survival rates of each plant after treating with a drug AZC, which causes the accumulation of insoluble abnormal proteins in these plants, it was confirmed that as the concentration of AZC became higher, the survival rate was lowered in the plant (Cas:DRR1) in which the function of the DRR1 gene is lost and the plants (DRR1-RNAi #1, #2) in which the function of the DRR1 gene is reduced, compared to the wild-type (WT) species, which is the control group (see Example 7).

These results of the embodiments of the present invention allow that the DRR1 gene is involved in drought stress tolerance and confirm that the DRR1 gene removes the insoluble proteins accumulated under the drought stress condition.

In addition, as another aspect of the present invention, the present invention provides a plant cell and a plant transformed with a composition for improving the tolerance of a plant to drought stress.

The preparation of the transgenic plant cell and transgenic plant of the present invention can be performed according to a method commonly known in the art. A plant can be transformed by inserting a foreign polynucleotide into a carrier (e.g., plasmids or viruses, etc.); by using Agrobacterium bacteria as a carrier, and by introducing a foreign polynucleotide directly into a plant cell. For example, in a case where using a vector that does not contain a T-DNA region is used, electroporation, microparticle bombardment, or polyethylene glycol-mediated uptake may be used.

Generally, in a method of transforming a plant, a method being frequently used is to infect a plant cell, seed, etc. with Agrobacterium tumefaciens, which is transformed with a foreign polynucleotide (U.S. Pat. Nos. 5,004,863, 5,349,124, and 5,416,011). Those skilled in the art can develop a transformed plant cell or seed into a plant by incubating or culturing the same under suitable conditions known in the art.

In addition, as still another aspect of the present invention, the present invention provides a method for improving tolerance of a plant to drought stress, which includes introducing into a plant cell, a composition for improving drought stress tolerance in plants, containing drought responsive ring 1 (DRR1) protein or a gene sequence encoding the protein.

Hereinafter, the present invention will be described in more detail through Examples. These Examples are only for describing the present invention in more detail, and it will be apparent to those skilled in the art that the scope of the present invention is not limited by these Examples according to the gist of the present invention.

### EXAMPLES

### Example 1. Isolation of DRR1 Gene

In order to select a novel RING-type E3 ligase involved in drought stress, the present inventors isolated and obtained the DRR1 gene involved in drought stress tolerance in plants from the cDNA of Arabidopsis thaliana by controlling the amount of insoluble proteins accumulated under the drying stress condition.

### Example 2. Confirmation of Increase of DRR1 Gene Under High Temperature and Drought Stress Conditions

In order to confirm whether the DRR1 gene is expressed under high temperature and drought stress conditions, RT-PCR was performed for each mRNA expression level under a high temperature stress condition of 37° C. and 42° C. and a drought stress condition using HSP17.4 as a marker gene for high heat stress and RD29A as a marker gene for drought stress. PCR was repeatedly performed for a total of 35 cycles with a change in temperature of 95° C.->55° C.->72° C. using DRR1-specific synthetic DNA nucleotide sequences (forward: 5′-GGGTGTTCGATTCTAGGTTTGG-3′ (SEQ ID NO: 3); reverse: 5′-GCAGAGAGGACAACAAGAGATGATAC-3′ (SEQ ID NO: 4)); and Taq-based polymerase.

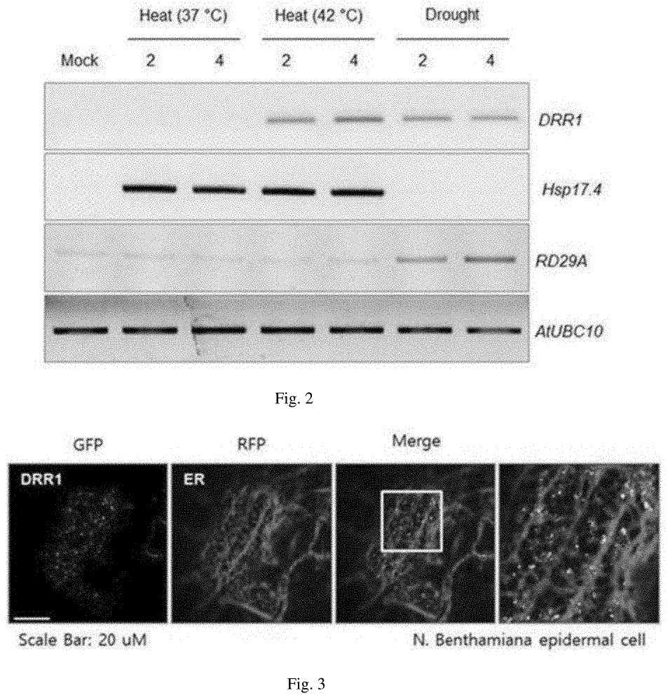

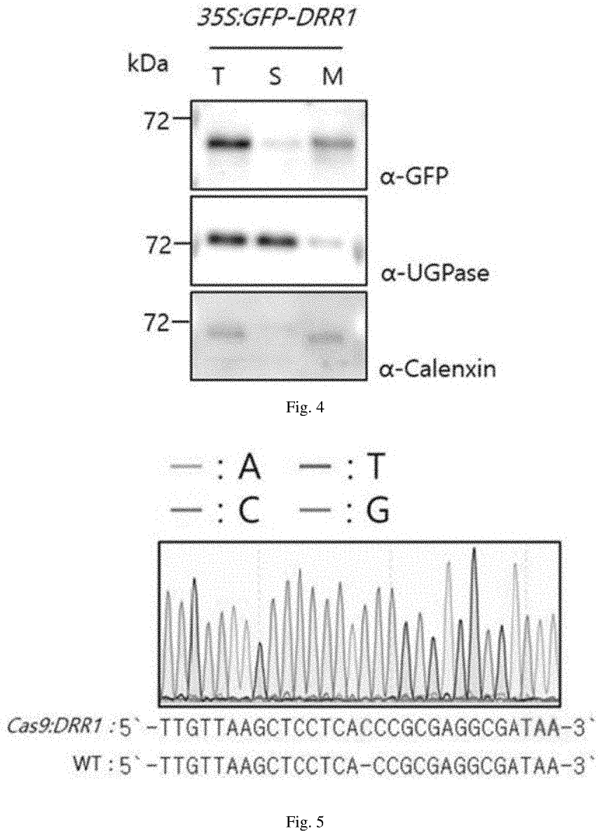

As a result, as shown in FIG. 2, it was confirmed that the expression level of the DRR1 gene was enhanced under high heat (42° C.) and drought stress conditions.

### Example 3. Confirmation of Intracellular Localization for Expression (Endoplasmic Reticulum) of DRR1 Gene

In order to confirm the intracellular localization of the DRR1 gene, the DRR1 gene was labeled with GFP and co-localization was performed in tobacco mesophyll cells together with the BiP1-mRFP-HDEL protein, which is a marker of the endoplasmic reticulum in plants.

As a result, as shown in FIG. 3, it was confirmed that when DRR1 labeled with GFP was expressed in tobacco mesophyll cells, it was expressed in connection with the endoplasmic reticulum labeled with BiP1-mRFP-HDEL.

In addition thereto, after expressing the DRR1-GFP recombinant protein in tobacco, ultracentrifugation was performed at 100,000×g (which is a condition for separation of the inner membrane) and the total solution (T), supernatant (S), and precipitate (M) were analyzed by fractionation assay, respectively, using UGPase as a cytoplasmic marker protein, and calnexin as a marker protein for the endoplasmic reticulum. From the above results, it can be inferred that DRR1 gene is located in the endoplasmic reticulum in the cell.

### Example 4. Preparation of Plant with a Loss of DRR1 Function and Plant with Reduced DRR1 Function

4-1. Preparation of Plant (Cas9:DRR1) with Reduced DRR1 Function Based on CRISPR-Cas9 Technology

A plant (Cas9:DRR1) in which the DRR1 gene sequence is modified was prepared based on the CRISPR-Cas9 technique, and the presence of loss of the function of the plant was confirmed.

As a result, as shown in FIG. 5, by sequencing the DRR1 CDS sequence, it was confirmed that due to the insertion of an additional cytosine between the 87th adenine and the 88th cytosine, the sequences from the 99th to the 101st (based on before cytosine insertion) are modified to TAA, a stop codon, resulting in an early stop.

4-2. Preparation of Plant (DRR1:RNAi) with Reduced DRR1 Function Based on RNAi Technology

A plant (DRR1:RNAi) in which the DRR1 gene sequence was modified based on the RNAi technique was prepared and the presence of reduction of the function of the plant was confirmed. In this case, UBC10 was used as a control for the same amount of samples.

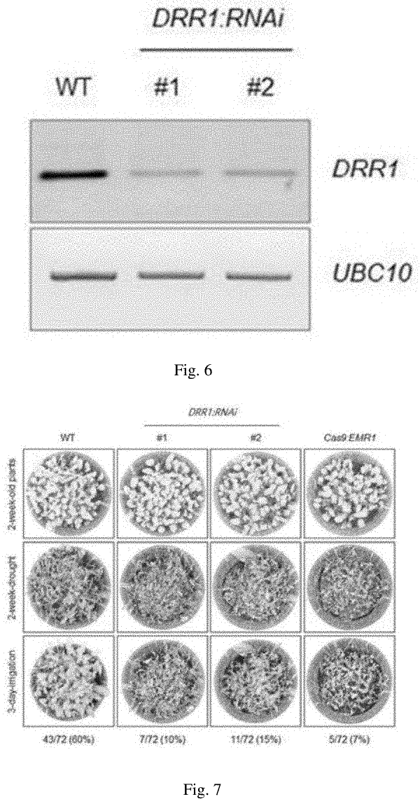

As a result, as shown in FIG. 6, it was confirmed that the mRNA of DRR1 was not detected in the plant in which the function of DRR1 was reduced by the RNAi technique.

### Example 5. Confirmation of Drought Stress Tolerance of Plant with a Loss of DRR1 Function and Plant with Reduced DRR1 Function

The plant (Cas:DRR1) with a loss of DRR1 function based on the CRISPR-Cas9 technique and the plants (DRR1-RNAi #1, #2) with reduction of DRR1 function based on the RNAi technique prepared in Example 4 were grown for two weeks (2-week-old plants), and then, a drought stress condition was induced without irrigation for two weeks (2-week-drought). In addition, each of the plants induced by drought stress was re-irrigated for three days (3-day-irrigation), and the tolerance of each plant to drought stress was examined.

As a result, when re-irrigation was performed as shown in FIG. 7, it was confirmed that while the plant of the wild-type (WT) species (the control group) showed a feature of recovery again, the plant (Cas:DRR1) with a loss of DRR1 function based on the CRISPR-Cas9 technique and the plants (DRR1-RNAi #1, #2) with reduction of DRR1 function showed much lower survival rates. From the above results, it can be confined that DRR1 is involved in drought stress tolerance.

### Example 6. Confirmation of Changes in Amount of Insoluble Protein Accumulation in Plant with a Loss of DRR1 Function and Plant with Reduced DRR1 Function Under Drought Stress Conditions

In order to confirm that DRR1 shows drought stress tolerance by inhibiting the accumulation of insoluble proteins under drought stress conditions, proteins were extracted from the plants (Cas:DRR1) with a loss of DRR1 function based on the CRISPR-Cas9 technique and the plants (DRR1-RNAi #1, #2) with reduction of DRR1 function in a normal state (Mock) and in the group treated with drought stress (Drought) without irrigation for 5 days, and the changes in the amount of an insoluble protein (ubiquitinated protein) were analyzed by centrifugation.

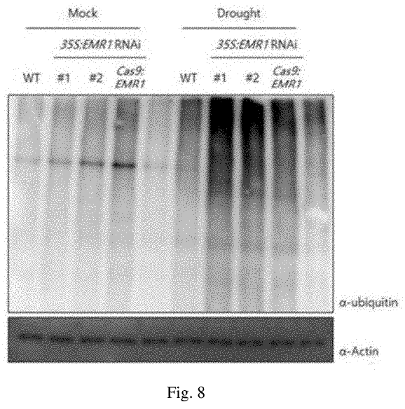

As a result, as shown in FIG. 8, it was confirmed that the amount of the insoluble protein was increased under the drought stress condition compared to the normal state (Mock), and particularly, it was confirmed that the amount of the ubiquitinated insoluble protein was increased in the plants (Cas:DRR1) with a loss of DRR1 function and the plants (DRR1-RNAi #1, #2) with reduction of DRR1 function compared to the wild-type (WT) species, which is the control group. From the above results, it can be inferred that DRR1 can remove insoluble proteins accumulated under drought stress conditions.

### Example 7. Confirmation of Insoluble Protein-Induced Stress Tolerance in Plant with a Loss of DRR1 Function and Plant with Reduced DRR1 Function

In order to confirm the tolerance to stress caused by an insoluble abnormal protein in the plant with a loss of DRR1 function and the plant with reduced DRR1 function, an azetidine-2-carboxylic acid (AZC) drug that causes the accumulation of insoluble proteins in plants was treated to the plants, and the survival rate of each plant was analyzed.

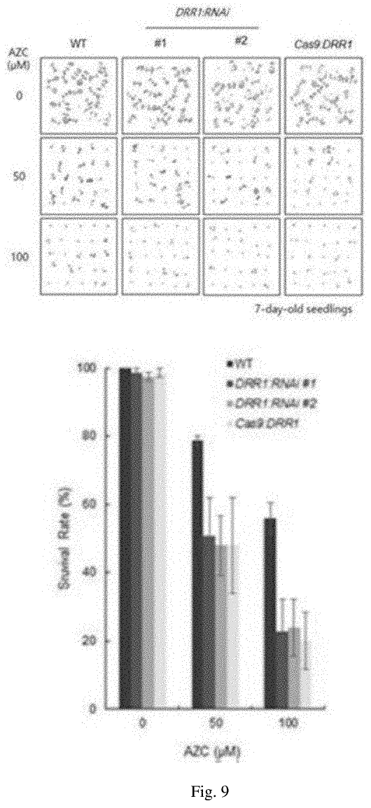

As a result, as shown in FIG. 9, it was confirmed that as the concentration of AZC became higher, the survival rates of the plant with a loss of DRR1 function and the plant with reduced DRR1 function were lowered, compared to the wild-type (WT) species, which is the control group. From the above results, it can be inferred that the reduced phenotypes of drought stress tolerance phenotype in the plant with a loss of DRR1 function and the plant with reduced DRR1 function are the result of not responding to the accumulation of insoluble proteins induced by drought stress.

The above embodiments of the present invention have been disclosed for illustrative purposes and those skilled in the art will appreciate that various modifications are possible, without departing from the technical spirit or essential features of the invention. Therefore, it should be understood that the embodiments described above are illustrative in all respects and not restrictive.

1: A method of improving tolerance of a plant to drought stress, the method comprising introducing into a plant cell a composition for improving drought stress tolerance in plants, the composition comprising drought responsive ring 1 (DRR1) protein or a gene sequence that encodes the protein.

2: The method of claim 1, wherein the drought responsive ring 1 (DRR1) protein comprises an amino acid sequence of SEQ ID NO: 1.

3: The method of claim 1, wherein the gene sequence that encodes the drought responsive ring 1 (DRR1) protein comprises a nucleotide sequence of SEQ ID NO: 2.

4: The method of claim 1, wherein the composition inhibits the accumulation of insoluble proteins in plants.

5: A plant cell having improved tolerance to drought stress, the cell being transformed with the method of claim 1.

6: A plant having improved tolerance to drought stress, the plant being transformed with the method of claim 1.

7. (canceled)


SEQUENCE LISTING (4 sequences)

SEQ ID NO: 1 - protein, 343 aa, Artificial Sequence
  DRR1 (Drought Responsive RING 1)
MNTRYSNQPELSSSNITITISSSALLSSSPRGDNSHVAAANGQERSPSSFYIRLAMKVSRARWFIFLRRVFHYQNGSRSDLGSNPFNSSTWMMSELIALLVQLTVITFTLAISKEERPIWPVRLWITGYDVGCLLNLMLLYGRYRQLDINQGNGFVLGDVEQQQRGREETRSSHLMNKCRTSLELFFAIWFVIGNVWVFDSRFGSFHHAPKLHVLCVSLLAWNAICYSFPFLLFLFLCCLVPLISSLLGYNMNMGSSDRAASDDQISSLPSWKFKRIDDSASDSDSDSATVTDDPECCICLAKYKDKEEVRKLPCSHKFHSKCVDQWLRIISCCPLCKQDLPR

SEQ ID NO: 2 - DNA, 1032 bp, Artificial Sequence
  DRR1 (Drought Responsive RING 1)
atgaatacacgttattccaatcagccggagttatcttctagtaatatcacgatcactatttcatcgtctgctttgttaagctcctcaccgcgaggcgataacagtcatgttgctgctgctaatggtcaagagaggtctccatcttcgttttatataaggctggctatgaaggtatctagagctagatggttcatcttcttgagaagagtgtttcactaccagaacggttcaagatctgaccttgggtctaatcctttcaattctagcacttggatgatgtctgagctcattgctctacttgttcagctcactgtgataacattcactctagctatctccaaagaagagagaccaatttggccagtgaggctatggatcacaggatacgatgtgggatgtcttttgaatctcatgctgttatatggtcggtatcgtcaactagacattaaccaaggaaatgggtttgtccttggcgatgttgagcagcaacagagaggcagagaagaaactaggtcctctcacttgatgaacaaatgcagaacgtcgctagagcttttctttgcgatttggtttgtgattggaaatgtttgggtgttcgattctaggtttggttctttccaccatgctcccaagcttcacgttctctgcgtctctcttttagcttggaacgctatctgctattcctttccctttcttctcttcctcttcctctgttgccttgttcctctcataagtagcctccttggatataacatgaacatgggatcctcagacagagcagcatcagatgaccaaatctctagtctccctagctggaaattcaaacgaatcgacgatagtgcttctgattctgattcagattcagctactgtaactgatgatccagagtgttgtatatgtttggcaaagtataaagacaaagaagaagtaaggaagcttccatgttcacataagtttcactcaaagtgtgtagatcaatggcttcgtatcatctcttgttgtcctctctgcaaacaagatcttccaagatga

SEQ ID NO: 3 - DNA, 22 bp, Artificial Sequence
  DRR1 specific forward primer
gggtgttcgattctaggtttgg

SEQ ID NO: 4 - DNA, 26 bp, Artificial Sequence
  Synthetic Construct
gcagagaggacaacaagagatgatac